(19) United States
(12) Patent Application Publication
(10) Pub. No.: US 2022/0133895 A1
(43) Pub. Date: May 5, 2022

(54) CAMPTOTHECIN-BASED DIMER COMPOUND, ANTICANCER DRUG AND METHOD OF ELIMINATING CANCER STEM CELL

(71) Applicant: HUAZHONG UNIVERSITY OF SCIENCE AND TECHNOLOGY, Hubei (CN)

(72) Inventors: Zifu LI, Hubei (CN); Xiangliang YANG, Hubei (CN); Jiankun GUAN, Hubei (CN)

(73) Assignee: HUAZHONG UNIVERSITY OF SCIENCE AND TECHNOLOGY, Hubei (CN)

(21) Appl. No.: 17/557,008

(22) Filed: Dec. 20, 2021

Related U.S. Application Data
(63) Continuation-in-part of application No. PCT/CN2021/098043, filed on Jun. 3, 2021.

(30) Foreign Application Priority Data
Oct. 30, 2020 (CN) 202011192519.6
May 31, 2021 (CN) 202110604561.2

(51) Int. Cl. A61K 47/55; A61K 31/4745; A61K 47/61; A61K 47/59; A61P 35/00

(52) U.S. Cl. CPC A61K 47/55; A61K 31/4745; A61P 35/00; A61K 47/61; A61K 47/593; A61K 47/551

(57) ABSTRACT

The disclosure provides a method for removing a cancer tumor stem cell and then further removing a tumor cell and provides an application of a drug molecule and its preparation in tumor treatment or prevention. On the one hand, by inducing the death of immunogenic cells, the anti-tumor immune response is enhanced. On the other hand, indoleamine-2,3-oxygenase is inhibited, immune cells, cytokines, amino acids, etc. are regulated, which improves the niche of cancer stem cells and makes them no longer conducive to the growth of cancer stem cells. Stem cell dormancy is lifted, and the sensitivity of cancer stem cells to chemotherapy drugs and immune cells is enhanced, so that cancer stem cells and tumor cells are effectively killed, and the efficacy of tumor treatment is improved.

### CROSS-REFERENCE TO RELATED APPLICATION

This application is a continuation-in-part of international PCT application serial no. PCT/CN2021/098043, filed on Jun. 3, 2021, which claims the priority benefit of China application no. 202110604561.2, filed on May 31, 2021, and China application no. 202011192519.6, filed on Oct. 30, 2020. The entirety of each of the above-mentioned patent applications is hereby incorporated by reference herein and made a part of this specification.

### TECHNICAL FIELD

The disclosure belongs to a cross field of a plurality of subjects including chemistry, pharmacy, medicine and the like, and in particular, relates to a camptothecin-based dimer compound, an anticancer drug, and a method of eliminating a cancer stem cell.

### DESCRIPTION OF RELATED ART

Tumors are the most critical factor threatening human health. At present, chemotherapy is one of the most effective methods for tumor treatment. Although most of the common chemotherapeutic drugs may bring specific therapeutic effects, a large number of chemotherapeutic drugs currently on the market still have significant shortcomings. Most chemotherapeutic drugs are organic small molecules with poor water solubility, so bioavailability of the chemotherapeutic drugs is low. Chemotherapy drugs are non-specifically and systemically distributed in the body. Therefore, while the chemotherapeutic drugs kill tumor cells, they also kill a large number of normal cells, causing serious toxic side effects. After decades of scientific and technological development, the nano drug-loading system has become one of the effective methods that can effectively solve the above-mentioned problems. At present, a variety of drug-loaded nano-preparations have entered the market, and a large number of nano-preparations are in clinical research and pre-clinical research stages.

Cancer stem cells are one of the main obstacles hindering the treatment of tumors. Clinical data shows that most tumors contain cancer stem cells. Chemotherapeutic drugs are currently the main means of treating various tumors. However, traditional chemotherapeutic drugs cannot effectively kill cancer stem cells and may increase the stemness of tumor cells, induce cancer stem cells to enter dormancy, and reduce their response to drugs and immune cells. Besides, the immunosuppressive microenvironment inside the tumor creates a cancer stem cell niche suitable for the growth and proliferation of cancer stem cells through immune cells, cytokines, and amino acids. How to effectively remove cancer stem cells is an important problem that needs to be solved urgently in clinical cancer treatment.

Camptothecin (CPT) is a cytotoxic quinoline alkaloid. Camptothecin can form a relatively stable “drug-topoisomerase 1-DNA” ternary complex with the topoisomerase 1-DNA cleavable complex through hydrogen bonds and intermolecular hydrophobic interactions. Thus, camptothecin can inhibit topoisomerase 1, exerting an antitumor effect. Camptothecin has significant anti-cancer properties, but the π-π interaction between the planar five-membered ring and the aromatic ring in its structure makes it low in solubility. Camptothecin is difficult to be administered systemically, has many adverse reactions, and has severe bone marrow suppression toxicity, which hinders the application of camptothecin in clinical practice. Besides, camptothecin can also cause an increase in immune regulatory T cells at the tumor site and inhibit the antitumor immune response. How to effectively improve the solubility of camptothecin, reduce its toxicity, and alleviate its promotion of immune regulatory T cells is an important issue that must be resolved whether camptothecin compounds can be used in clinical applications.

The amphiphilic polymer drug-loading nanosystem is one of the most researched nano-drug-loading systems. This system may provide a hydrophobic core to solubilize hydrophobic drug molecules, and its hydrophilic shell may reduce protein adsorption, reduce the phagocytic clearance of the reticuloendothelial system, and extend the half-life of the drug in the body. Further, in this system, certain receptors highly expressed in tumor tissues are used to modify their specific ligands in the amphiphilic polymer drug-loading nanosystem. In this way, the tumor targeting of the drug may be significantly improved, systemic side effects may be reduced, and the antitumor efficacy may be enhanced. At present, polyethylene glycol is the most hydrophilic molecule used in amphiphilic polymer drug-loading nanosystems, and drug-loading nano-preparations based on polyethylene glycol have hit the market. However, in the course of clinical use, a large number of studies have found that the use of polyethylene glycol as a drug-loading nano-preparation of hydrophilic surface molecules may easily induce the production of antibodies that specifically eliminate the drug-loading nanosystem in patients and produce tolerance.

The replacement of polyethylene glycol with hydroxyethyl starch of natural origin and better biocompatibility is expected to solve the above problems. Currently, there are few reports on the preparation of amphiphilic drug-loading nanosystems using hydroxyethyl starch as a hydrophilic fragment. In the early stage of this research group, a nano drug-loading system based on amphiphilic hydroxyethyl starch coupled with polylactic acid copolymer is reported. The long-term circulation of the drug-loading nanosystem in the blood system of the body may be realized without causing the body's immune rejection reaction. However, poor tumor targeting is provided, so a large number of drug-loading nanoparticles accumulate in non-tumor sites, resulting in specific side effects and reduced antitumor activity.

On the other hand, although the existing amphiphilic polymer drug-loading nanosystems can improve the therapeutic effect to a certain extent, the drug dosage is large most of the time. Chemotherapy drugs are known to kill tumor cells, but also have certain greater toxic side effects on normal cells. Therefore, how to reduce the dosage of drugs while improving the tumor treatment effect of the nano drug-loading system is also an urgent technical problem in this field.

### SUMMARY

Regarding the technical defects found in the related art, the disclosure aims to provide a camptothecin-based dimer compound, an anticancer drug, and a method of eliminating cancer stem cell. Experiments show that the dimer compound may improve the solubility of camptothecin, reduce its toxicity, alleviate the promotion effect of camptothecin on regulatory T cells, and reverse the drug resistance of cancer stem cells caused by camptothecin. The nano drug-carrying system based on the dimer compound and the polylactic acid-hydroxyethyl starch-folate macromolecular compound may maintain long-term stability in the blood environment. Further, Experiments have found that the camptothecin-based dimer compound and the nano drug-loading system based on the compound provided by the disclosure may induce immunogenic cell death and inhibit indoleamine-2,3-oxygenase. In this way, the niche of cancer stem cells is abolished, the dormancy of cancer stem cells is relieved, the purpose of eliminating cancer stem cells is achieved, and the antitumor efficacy is improved. The technical problems of the nano drug-loading system provided by the related art such as low targeting ability, large drug dosage, low antitumor activity, and significant toxicity and side effects may thereby be solved.

In order to achieve the above objective, according to the first aspect of the disclosure, a camptothecin-based dimer compound is provided, which has a structural formula as shown in formula (1):

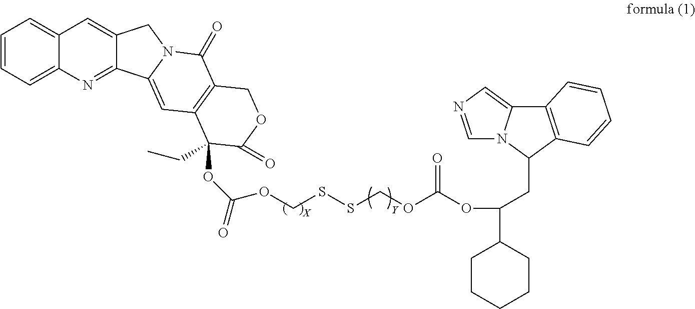

where x is an integer of 1 to 4, and y is an integer of 1 to 4.

According to the secondaspect, an anticancer drug is further provided, and the anticancer drug includes the abovementioned dimer compound and a pharmaceutically acceptable additive.

In a preferred embodiment, the anticancer drug includes a nano drug-loading system and a pharmaceutically acceptable additive. The nano drug-loading system includes the amphiphilic polylactic acid-hydroxyethyl starch-folate macromolecular compound and an antitumor drug, and the antitumor drug is the dimer compound. The macromolecular compound is obtained by coupling folic acid and polylactic acid with hydroxyethyl starch through an ester bond and has a structural formula shown in formula (3):

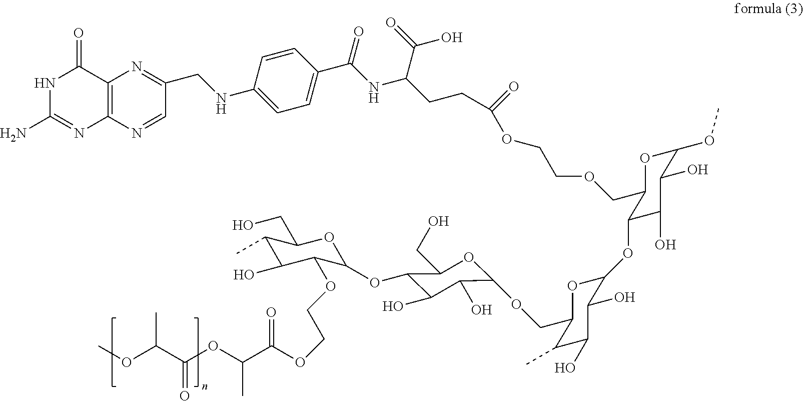

A molecular weight of the polylactic acid is 6 KDa to 10 KDa, and a molecular mass of the hydroxyethyl starch is 100 KDa to 150 KDa. A grafting rate of the polylactic acid on the hydroxyethyl starch is 0.5 to 1. A grafting rate of the folic acid on the hydroxyethyl starch is 10 to 30. In a preferred embodiment, in the structural formula of the dimer compound, x=2 and y=2.

In a preferred embodiment, a preparation method of the nano drug-loading system based on the polylactic acid-hydroxyethyl starch-folate macromolecular compound includes the following steps.

(1) The amphiphilic polylactic acid-hydroxyethyl starch-folate macromolecular compound is dissolved in water, ultrasonic emulsification is performed in an ice bath, and an organic solution in which the dimer compound shown in formula (1) is dissolved is simultaneously added to obtain an ultrasonic emulsion.

(2) An organic solvent of the emulsion obtained in step (1) is removed through reduced pressure distillation to obtain the amphiphilic polylactic acid-hydroxyethyl starch-folate macromolecular compound-based nano drug-loading system containing the dimer compound drug.

According to the third aspect, a method for removing a cancer stem cell and a tumor cell is provided. In the method, dormancy of a cancer stem cell is relieved through improving a cancer stem cell niche to eliminate the cancer stem cell and improve antitumor efficacy.

In a preferred embodiment, immunogenic cell death is induced, and indoleamine-2,3-oxygenase is inhibited to improve the cancer stem cell niche.

Preferably, one or more of chemotherapy, radiotherapy, hyperthermia, cryotherapy, magnetic therapy, sound waves, laser, and electrical stimulation is performed to induce immunogenic cell death.

One or more of the following: inhibiting expression of indoleamine-2,3-oxygenase, inhibiting activity of indoleamine-2,3-oxygenase, inhibiting a downstream pathway of indoleamine-2,3-oxygenase, and reducing a metabolite of indoleamine-2,3-oxygenase by using an anticancer drug is performed, to inhibit an indoleamine-2,3-oxygenase pathway and to abolish the cancer stem cell niche and ultimately eliminate the cancer stem cell and the tumor cell.

In a preferred embodiment, the anticancer drug includes the camptothecin-based dimer compound.

To sum up, the above technical solutions provided by the disclosure have the following beneficial effects compared with the related art.

(1) The disclosure provides a camptothecin-based dimer prodrug compound, which couples camptothecin and NLG919 by using reduction-responsive dithiodiol substances. By evaluating the antitumor activity of the compound, it is found that it shows good cell killing activity against tumor cells such as breast cancer, liver cancer, ovarian cancer, colon cancer, and melanoma. Further, the dimer prodrug compound and the first-line drug Irinotecan are used in animal experiments, and the results show that the dimer prodrug compound provided by the disclosure has a better antitumor effect than the camptothecin-type marketed drug Irinotecan and the first-line clinical breast cancer drug Taxol as the tumor volume is significantly reduced, and the tumor weight is significantly reduced.

(2) In the camptothecin-based dimer prodrug compound provided by the disclosure, the solubility of camptothecin is enhanced, and the toxicity of camptothecin is significantly reduced. The reduction responsiveness of this compound is evaluated by mass spectrometry. The compound may be completely degraded into the original camptothecin and NLG919 under the action of reducing substances such as glutathione after entering the tumor cells, which is beneficial for the compound to play a corresponding role in the tumor cells.

(3) Compared with NLG919, the inhibitory activity of the dimer compound of camptothecin and NLG919 provided by the disclosure on IDO is greatly improved. When the drug concentration of NLG919 is about 8 μm, the inhibition rate of IDO is 50%, while the dimer compound IDO provided by the disclosure only needs approximately 0.08 μm to reach the inhibition rate of 50%. The possible reason is that the dimer of the disclosure is easier to combine with IDO and inhibit the activity of IDO.

(4) The dimer compound provided by the disclosure has been proved to be able to significantly improve the tumor immune microenvironment and significantly increase the sensitivity of cancer stem cells to chemotherapeutic drugs.

(5) In the disclosure, a reduction-responsive intermediate linking reagent containing disulfide bonds is particularly used to couple camptothecin and NLG919 to obtain a dimer compound, and the tumor cells contain high concentrations of reducing glutathione. The dimer compound provided by the disclosure may be reduced to the original camptothecin and NLG919 under reducing conditions, and exerts a tumor-killing effect and an IDO inhibitory effect, respectively. The tumor is killed and the tumor immune microenvironment is also regulated, and the effect of tumor treatment is thereby enhanced, the combination of tumor chemotherapy and immunotherapy is achieved, and the antitumor effect is improved. Further, experiments have proved that its tumor suppression effect is better than that brought by the mature first-line drug Irinotecan and paclitaxel injection Taxol currently available on the market, and therefore, the nano drug-loading system provided by the disclosure may be used as a lead compound for the development of antitumor drugs and exhibits good application prospects.

(6) The nano drug-loading system based on the polylactic acid-hydroxyethyl starch-folate macromolecular compound provided by the disclosure shows better antitumor activity than free drugs under the same dosage. The tumor volume and weight of breast cancer are significantly inhibited, the proliferation of tumor cells in breast cancer is significantly inhibited, the apoptosis of tumor cells is promoted, and the survival time of breast cancer mice is significantly prolonged.

(7) In the nano drug-loading system based on the polylactic acid-hydroxyethyl starch-folate macromolecular compound provided by the disclosure, the drug CN is obviously enhanced to induce the death of immunogenic cells (ICD) and inhibit the activity of indoleamine-2,3-oxygenase (IDO). Further, the maturation of lymph node dendritic cells (DC) is promoted, the number of effector memory T cells in the spleen increases, and the central memory T cells are maintained high. The tumor microenvironment is improved, the proportion of suppressive T cells in the tumor is reduced, the content of tryptophan is increased, and the concentration of IL-6, IL-13, and TGF-β is reduced, so that the elimination of cancer stem cells is thereby achieved.

(8) In the nano drug-loading system based on the polylactic acid-hydroxyethyl starch-folate macromolecular compound, the drug may be delivered to the tumor site in a targeted manner. The tumor is killed and the tumor immune microenvironment is also regulated, and the effect of tumor treatment is thereby enhanced, the combination of tumor chemotherapy and immunotherapy is achieved, and the antitumor effect is improved. Further, experiments have proved that its tumor suppression effect is better than that brought by the mature first-line drug Irinotecan and paclitaxel injection Taxol currently available on the market, tumor volume and tumor weight are lower than half of that treated with Irinotecan and Taxol, and therefore, the nano drug-loading system provided by the disclosure exhibits good application prospects.

(9) In the polylactic acid-hydroxyethyl starch-folate macromolecular compound provided by the disclosure, folic acid and polylactic acid are coupled with the hydroxyethyl starch through an ester bond to obtain an amphiphilic macromolecule, and the molecule has good biocompatibility. Through the adoption of the emulsification solvent evaporation method, the anti-tumor small molecule CN is encapsulated into the hydrophobic core of the polymer nanoparticles to form drug-loading nanoparticles with a particle size of approximately 200 nm and a uniform distribution. The drug-loading nanoparticles have good stability, may circulate in the blood system for a long time, and may quickly and specifically enrich the tumor site through the targeting effect of folic acid. Under the mediation of folate receptors, tumor cells take up drug-loading nanoparticles into cells. Under the action of reducing substances in tumor cells, part of the drug CN is reduced to CPT and NLG919, which promotes the depolymerization of nanoparticles. The depolymerization of nanoparticles in turn promotes the release of drugs, thereby killing tumors and inhibiting indoleamine-2,3-oxygenase (IDO) activity. On the one hand, the drug CN and its reduction product CPT promote the maturation of dendritic cells and their antigen presentation by inducing immunogenic cell death, and anti-tumor immune response and anti-tumor immune memory are thereby triggered. On the other hand, the drug CN and its reduction product NLG inhibit IDO, inhibit the conversion of tryptophan to kynurenine, and maintain a high concentration of tryptophan in the tumor microenvironment. As such, the proportion of regulatory T cells is reduced, and the content of immunosuppressive cytokines IL-6, IL-13, and TGF-β is reduced, and the immunosuppression in the tumor microenvironment is thereby alleviated. In both positive and negative aspects, the anti-tumor immune response is enhanced, the cancer stem cell niche is abolished, and the dormant cancer stem cells are released from cycle inhibition. Thus, the sensitivity of cancer stem cells to immune cells and chemotherapeutic drugs is enhanced, and the effective elimination of cancer stem cells and tumor cells is finally realized. In summary, in the drug-loading nano-system based on polylactic acid-hydroxyethyl starch-folate loaded with the drug CN, good anti-tumor effects are achieved in a variety of mouse triple-negative breast cancer models, and the survival time of the mice is significantly prolonged.

(10) The disclosure further provides a method for removing a cancer stem cell and then further removing a tumor cell. By inducing the death of immunogenic cells and simultaneously inhibiting indoleamine-2,3-oxygenase, the cancer stem cells are eliminated. Experiments have showed that through this method, cancer stem cell niches may be effectively abolished, cancer stem cell dormancy is alleviated, cancer stem cells are eliminated, and good anti-tumor efficacy is achieved.

(11) In the method of eliminating a cancer stem cell provided by the disclosure, in an embodiment, a prodrug molecule of a dimer compound based on camptothecin is designed and prepared. Experiments have found that the prodrug may significantly induce immunogenic cell death and significantly inhibit the activity of indoleamine-2,3-oxygenase. The inhibition of the G1/S cycle of cancer stem cells is thereby released, the apoptosis of cancer stem cells is promoted, and the proliferation and cloning ability of cancer stem cells is reduced.

(12) In a method of eliminating a cancer stem cell provided by an embodiment of the disclosure, a prodrug molecule CN and its nano dosage form CN@PHF are further designed and prepared. Experiments have found that the nano dosage form may significantly induce immunogenic cell death, significantly inhibit the activity of indoleamine-2,3-oxygenase, significantly regulate various immune cells, cytokines, amino acids, etc. inside the tumor, abolish the niche of cancer stem cells, relieve stem cell dormancy, eliminate cancer stem cells, and significantly inhibit tumor growth and prolong survival time.

(13) The disclosure provides a method for removing a cancer stem cell and then further removing a tumor cell. By inducing immunogenic cell death and inhibiting indoleamine-2,3-oxygenase, the purpose of eliminating cancer stem cells may be achieved. Based on the above, according to this mechanism, drugs and their formulations are designed to kill tumors while regulating cancer stem cell niches, enhancing tumor treatment effects, realizing the combination of tumor chemotherapy and immunotherapy, and enhancing anti-tumor efficacy. Experiments have proved that the tumor suppressive effect of the treatment by this method is better than the current mature first-line drug Irinotecan and paclitaxel injection Taxol. Therefore, the method for removing stem cells provided by the disclosure and the drug molecules and dosage forms designed by the method have good application prospects.

### BRIEF DESCRIPTION OF THE DRAWINGS

FIG. 1 is a flow chart of preparation of a compound CN in Example 1 according to the disclosure.

FIG. 2 is an H NMR spectrum (600M) of the compound CN prepared in Example 1 according to the disclosure.

FIG. 3 is an ultraviolet spectrum (content a) and infrared spectrum (content b) of the compound CN, camptothecin, and NLG prepared in Example 1 according to the disclosure.

FIG. 4 is a mass spectrum of a reduced product of the compound CN prepared in Example 1.

FIG. 5 is a graph of in vitro IDO activity inhibition of the compound CN prepared in Example 1 according to the disclosure.

FIG. 6 is in vitro activity evaluation of CN inducing immunogenic cell death in Example 4 according to the disclosure.

FIG. 7 is the evaluation of CN in vivo anti-tumor activity carried out in Example 5 according to the disclosure.

FIG. 8 is a functional evaluation of CN in vivo to stimulate anti-tumor immunity and relieve immunosuppression in Example 6 according to the disclosure.

FIG. 9 shows the effect of CN performed in Example 7 on the ability of cancer stem cells to dormant, survive, and proliferate to form clonal spheres according to the disclosure.

FIG. 10 is an H NMR spectrum (600M) of a compound PHF prepared in Example 8 according to the disclosure.

FIG. 11 is particle size morphology and blood stability of a nano-preparation CN@PHF prepared in Example 8 according to the disclosure.

FIG. 12 is an evaluation of antitumor activity of the nano-preparation CN@PHF in Example 9 according to the disclosure.

FIG. 13 is an evaluation of the activity of the nano-preparation CN@PHF in the in vivo induction of immunogenic cell death, suppression of regulatory T cells, and elimination of cancer stem cells in Example 10 according to the disclosure.

FIG. 14 is the evaluation of the activity of the nano-preparation CN@PHF in enhancing tumor immunity in vivo in Example 10 according to the disclosure.

FIG. 15 is the evaluation of the activity of the nano-preparation CN@PHF in improving the niche of cancer stem cells in vivo in Example 11 according to the disclosure.

FIG. 16 shows effects of the nano-preparation CN@PHF performed in Example 12 on the survival time of tumor-bearing mice according to the disclosure.

### DESCRIPTION OF THE EMBODIMENTS

In order to make the objectives, technical solutions, and advantages of the disclosure clearer and more comprehensible, the disclosure is further described in detail with reference to the drawings and embodiments. It should be understood that the specific embodiments described herein serve to explain the disclosure merely and are not used to limit the disclosure.

The disclosure provides a camptothecin-based dimer compound including a structural formula as shown in formula (1):

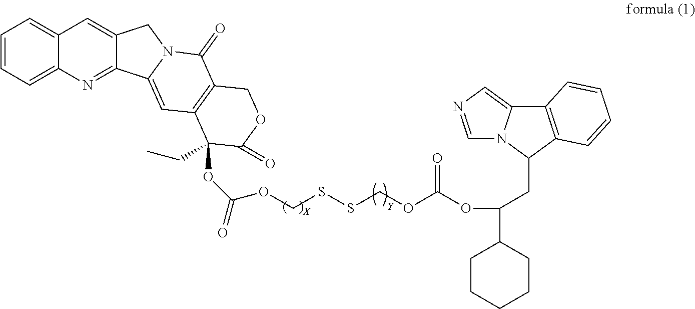

where x is an integer of 1 to 4 and y is an integer of 1 to 4, and preferably, x is an integer of 1 to 3 and y is an integer of 1 to 3.

In a preferred embodiment, in the structural formula of the dimer compound, x=2 and y=2. That is, the structural formula of the preferred compound is as shown in formula (2) (the compound is abbreviated as CN in the specification):

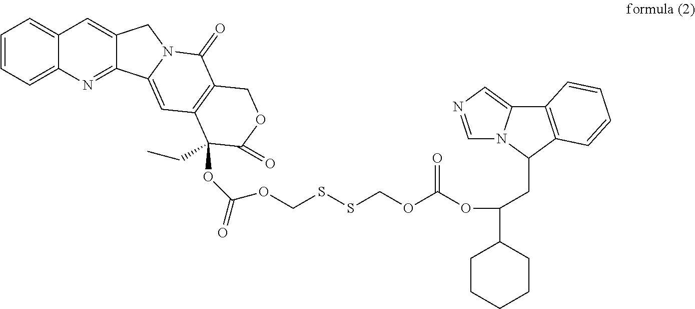 [SMILES: CC[C@@]1(OC(=O)OCSSCOC(=O)OC(CC2c3ccccc3-c3cncn32)C2CCCCC2)C(=O)OCc2c1cc1n(c2=O)Cc2cc3ccccc3nc2-1]

The disclosure further provides a preparation method of the dimer compound, and the method includes the following steps.

(1) A hydroxy group in a camptothecin structural formula undergoes substitution reaction and is converted into a phosgene compound. The phosgene compound and disulfide bond-containing alkyl diol then undergo an esterification reaction, so that the camptothecin and the disulfide bond-containing alkyl diol are connected through a carbonate bond to obtain an intermediate product.

(2) After the intermediate product in step (1) is separated and purified, the hydroxyl group contained in its structure undergoes substitution reaction and is converted into an activated product. The activated product is a phosgene compound. The phosgene compound and NLG919 then undergo a substitution reaction, so that the intermediate product and NLG919 are connected through a carbonate bond. The obtained product is separated and purified to obtain the dimer compound.

In some embodiments, step (1) specifically includes the following steps. The camptothecin, an acid chloride reagent, and an acid binding agent are mixed and dissolved in an organic solvent after mixing, so that the hydroxyl group in the camptothecin structural formula reacts with the acid chloride reagent to generate a phosgene compound. The phosgene compound and the disulfide bond-containing alkyl diol undergo a substitution reaction, so that the camptothecin and the disulfide bond-containing alkyl diol are connected through a carbonate bond to obtain the intermediate product. Step (2) specifically includes the following steps. The intermediate product in step (1), the acid chloride reagent, and the acid binding agent are mixed and are dissolved in an organic solvent after mixing to cause the hydroxyl group contained in the structure to undergo a substitution reaction and be converted into a phosgene compound. The phosgene compound and NLG919 undergo a substitution reaction, so that the intermediate product and NLG919 are connected through carbonate to obtain the dimer compound.

In some embodiments, the acid chloride reagent is phosgene and/or triphosgene. The organic solvent is one or more of dichloromethane, chloroform, or tetrahydrofuran, and the acid binding agent is one or more of 4-dimethylaminopyridine, pyridine, or triethylamine. The organic solvent is used to dissolve camptothecin and the acid chloride reagent, and the acid binding agent is also used as a catalyst and may be used to catalyze the decomposition of triphosgene.

In some embodiments, the intermediate product described in step (1) is separated and purified by the following steps. The intermediate product is extracted with a dilute acid aqueous solution, saturated brine, and ultrapure water in sequence to extract a reaction product (the unreacted catalyst, acid chloride reagent, and disulfide bond-containing alkyl diol are removed). An organic phase is obtained through separation. Water is removed first with a desiccant, the organic solvent is then removed, and finally the obtained organic phase is dried to obtain the intermediate product.

In some embodiments, the separation and purification in step (2) includes the following steps. The product obtained in step (2) is extracted with the dilute acid aqueous solution, saturated brine, and ultrapure water in sequence to remove raw materials not participating in the reaction. The organic phase is obtained by separation. The water is removed with the desiccant first, and the organic solvent is then removed to obtain a crude product. The product is purified by HPLC and finally dried to obtain the dimer compound.

In some embodiments, the disulfide bond-containing alkyl diol is one or more of 2,2′-dithiodiethanol, dithiodimethanol, 3,3′-dithiodipropanol, or 4,4′-dithiodibutanol.

Steps (1) and (2) in the disclosure may be reacted in a wide temperature range, such as −5° C. to 30° C., and in order to avoid the conversion of phosgene to the gas phase, the reaction may selectively be carried out on ice in some embodiments. A theoretical reaction molar ratio of the camptothecin, disulfide bond-containing alkyl diol, and NLG919 is 1:1:1, and a material-adding ratio may be close to this ratio. The acid binding agent is also used as a catalyst, and its amount may be equivalent to that of the camptothecin. The amount of the acid binding agent is 3 to 4 times that of the acid chloride reagent phosgene or triphosgene, and the organic solvent is used to dissolve raw materials.

In the disclosure, the application of the dimer compound in the treatment of cancer is also provided.

In some embodiments, the dimer compound is used to eliminate cancer stem cells and cancer tumor cells. Therefore, it is equivalent that a method for removing cancer stem cells and tumor cells is also provided in this embodiment.

In some embodiments, the cancer is breast cancer, liver cancer, colon cancer, ovarian cancer, or melanoma.

An anticancer drug is also provided in this embodiment, and the anticancer drug includes the abovementioned dimer compound and a pharmaceutically acceptable additive. In some embodiments, a dosage form of the anticancer drug is injection, powder injection, oral preparation, spray, capsule, or suppository.

Indoleamine 2,3-dioxygenase (IDO) is an important negative feedback regulator enzyme that supports tumor cell growth in a tumor immunosuppressive microenvironment. IDO-mediated degradation of tryptophan to kynurenine is one of the important mechanisms for tumor cell immune escape. IDO expressed by tumor cells and antigen-presenting cells may catalyze the degradation of the essential amino acid tryptophan and produce a large number of metabolites. The activity of tumor-specific effector CD8+ T cells is thereby suppressed, and at the same time, the immunosuppressive activity and number of regulatory T cells (Treg) are enhanced. Clinical studies have reported that the high expression of IDO in tumors is associated with poor prognosis and tumor drug resistance. NLG919 (CAS number: 1402836-58-1) is a selective inhibitor of IDO with EC50 of 75 nM. In some embodiments of the disclosure, reduction-responsive 2,2′-dithiodiethanol is used to couple camptothecin with NLG919. Spectral analysis methods and other means may be applied to determine its specific structure as shown in formula (2), and the compound is named CN in the disclosure. The reduction responsiveness of this compound is evaluated by mass spectrometry, and it can be completely degraded into the original camptothecin and NLG919. By evaluating the antitumor activity of the compound, it is found that it shows good cell killing activity against tumor cells such as breast cancer, liver cancer, ovarian cancer, colon cancer, and melanoma. Moreover, the inhibitory activity of this compound on IDO is greatly improved compared with NLG919. In addition, the compound CN may significantly increase the sensitivity of cancer stem cells to chemotherapeutic drugs. The compound CN may be used as a lead compound for the development of antitumor drugs.

In the disclosure, the reducing disulfide bond is connected to camptothecin and the small immunomodulatory molecule NLG919, the combination of tumor chemotherapy and immunotherapy may thereby be achieved, and the antitumor efficacy may be enhanced. The compound CN prepared by the disclosure may be reduced to the original camptothecin and NLG919 under reducing conditions, and exerts good anti-tumor activity in tumors such as liver cancer, breast cancer, colon cancer, melanoma, and ovarian cancer. By inhibiting the activity of indoleamine 2,3 oxygenase, DC cell maturation is increased, the number of immunosuppressive Treg cells is decreased, cancer stem cell dormancy is lowered, and tumor immune response is enhanced. The compound provided by the disclosure requires a simple preparation method and mild conditions and exhibits strong reproducibility and good activity.

Regarding a polylactic acid-hydroxyethyl starch-folate macromolecular compound (abbreviated as PLA-HES-FA in the disclosure, further abbreviated as PHF) in a drug carrier for the anticancer drug of the dimer compound, the macromolecular compound is obtained by coupling folic acid and polylactic acid with hydroxyethyl starch through an ester bond. It has the structural formula shown in formula (3):

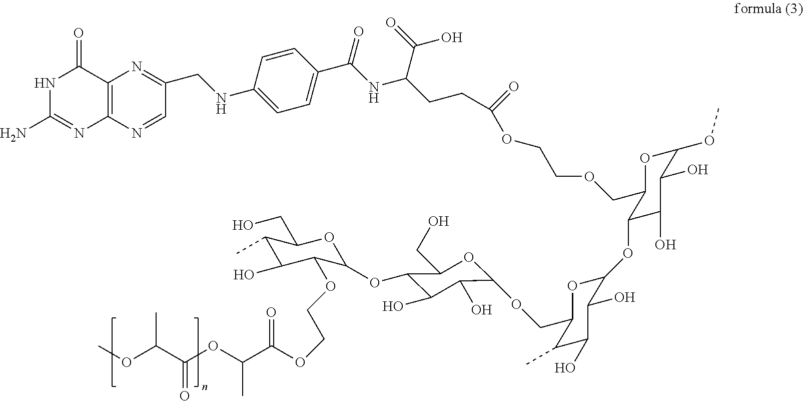

where a molecular weight of the polylactic acid is 6 KDa to 10 KDa (the corresponding range of n is approximately 97-161), and a molecular weight of the hydroxyethyl starch is 100 KDa to 150 KDa. A grafting rate of the polylactic acid on the hydroxyethyl starch is 0.5 to 1, and preferably 0.7 to 0.9. A grafting rate of the folic acid on the hydroxyethyl starch is 10 to 30, and preferably 15 to 25.

In a preferred embodiment, the molecular weight of the polylactic acid is 8 KDa, and the molecular weight of the hydroxyethyl starch is 130 KDa.

The disclosure further provides a preparation method of the macromolecular compound, and the method includes the following steps.

(1) The carboxyl group of the folic acid and the hydroxyl group of the hydroxyethyl starch undergo an esterification reaction to obtain an intermediate product.

(2) After the intermediate product in step (1) is separated and purified, the hydroxyl group of the intermediate product and the carboxyl group of polylactic acid undergo an esterification reaction. The obtained product is separated and purified to obtain the macromolecular compound.

In some embodiments, step (1) specifically includes the following steps. Folic acid, a carboxyl activating reagent, and an acid binding agent are mixed and dissolved in an organic solvent after mixing to make the carboxyl group in the folic acid structural formula react with the carboxyl activating reagent to form an activated ester. The activated ester and the hydroxyl-containing hydroxyethyl starch then undergo a substitution reaction, so that the folic acid and the hydroxyl-containing hydroxyethyl starch are connected through an ester bond, and the intermediate product is obtained. The carboxyl activating reagent is dicyclohexylcarbodiimide or 1-ethyl-3(3-dimethylpropylamine)carbodiimide, the organic solvent is dimethyl sulfoxide and/or tetrahydrofuran, and the acid binding agent is one or more of 4-dimethylaminopyridine, pyridine, and triethylamine. The organic solvent is used to dissolve folic acid, polylactic acid, hydroxyethyl starch, and carboxyl activating reagent, and the acid binding agent is also used as a catalyst and may be used to catalyze the substitution reaction.

In some embodiments, step (2) specifically includes the following steps. The polylactic acid, carboxyl group activating reagent, and acid binding agent are mixed and dissolved in an organic solvent after mixing, so that the carboxyl group contained in the polylactic acid structure is substituted and converted to an activated ester. The activated ester and the intermediate product described in step (1) then undergo a substitution reaction and are connected through an ester bond to obtain the macromolecular compound.

In some embodiments, the intermediate product described in steps (1) and (2) is separated and purified through the following steps. The obtained intermediate product is precipitated with ethanol, dialyzed with ultrapure water after being precipitated and dissolved, and finally freeze-dried to obtain the intermediate product.

In some embodiments, the intermediate product described in step (1) is separated and purified through the following steps. The obtained intermediate product is precipitated with ethanol, dialyzed with ultrapure water after being precipitated and dissolved with dimethyl sulfoxide, and finally freeze-dried to obtain the intermediate product in sequence.

Steps (1) and (2) in the disclosure may be reacted in a wide temperature range, such as 20° C. to 60° C. The material-feeding molar ratio of the polylactic acid, hydroxyethyl starch, and folic acid is (2-6):1:(10-30), preferably (3-5):1:(15-25), and more preferably 4:1:20. Experiments show that an excessively large grafting rate of folic acid may reduce the solubility of the prepared macromolecular compound. The acid binding agent is also used as a catalyst, and its amount may be equivalent to the amount of the folic acid or polylactic acid, and the organic solvent is used to dissolve the raw material.

The application of the macromolecular compound in the preparation of a nanomedicine drug-loading system is also provided, and the nanomedicine drug-loading system is used for the treatment of cancer.

In some embodiments, the cancer is breast cancer, liver cancer, colon cancer, ovarian cancer, or melanoma.

The disclosure also provides a nano drug-loading system based on the polylactic acid-hydroxyethyl starch-folate macromolecular compound. The nano drug-loading system includes the amphiphilic polylactic acid-hydroxyethyl starch-folate macromolecular compound and the antitumor drug. The antitumor drug is the camptothecin-based dimer compound as shown in formula (1), and more preferably the compound CN as shown in formula (2).

The hydroxyethyl starch raw material in the embodiments of the disclosure was purchased from Wuhan Huake University Life Technology Co., Ltd. The molecular weight of the hydroxyethyl starch used was 130 KDa, and the degree of substitution with hydroxyethyl was 0.4. The polylactic acid raw material of in the disclosure was purchased from Jinan Daigang Bioengineering Co., Ltd., and the molecular weight of the polylactic acid was 8 KDa.

The drug CN in a preferred embodiment of the disclosure is the camptothecin-based dimer compound as described in formula (3) with a purity of 99%.

Hydroxyethyl starch (HES) is a modified natural polysaccharide, which is a product obtained by acid hydrolysis of highly branched starch and reaction with ethylene oxide. Hydroxyethyl starch is mainly used as a plasma expander in clinical practice, and it is also the first choice for the treatment of hypovolemia and shock. Hydroxyethyl starch has good biocompatibility, and it has a lower incidence of hypersensitivity than glucose. Hydroxyethyl starch also has good water solubility and biocompatibility and may be degraded by α-amylase in the body and excreted from the urine through glomerular filtration. In view of the above-mentioned advantages of hydroxyethyl starch, by treating the hydroxyethyl starch as an amphiphilic polymer nano drug-loading system, a nano drug-loading system that can achieve long-term stable circulation in the body may be obtained, and the half-life of the drug is improved. Breast cancer cells show high expression of folate receptors, and folic acid may specifically bind to the folate receptors on the surface of tumor cells. Therefore, by modifying the surface of the nanoparticle with folic acid, tumor-specific targeted delivery of nanomedicine may be realized, and the uptake of the drug-loaded nanoparticle by tumor cells may be improved.

In some embodiments, a size of a drug-loaded nanoparticle of the nano drug-loading system is 100 nm to 200 nm. A maximum drug loading capacity of the nano drug-loading system may reach 15%.

The disclosure further provides a preparation method of the nano drug-loading system based on the drug carrier, and the method includes the following steps.

(1) The amphiphilic polylactic acid-hydroxyethyl starch-folate macromolecular compound is dissolved in water, ultrasonic emulsification is performed in an ice bath, and a dichloromethane solution in which the dimer compound shown in formula (1) is dissolved is simultaneously added to obtain an ultrasonicated emulsion.

(2) The emulsion obtained in step (1) is placed in a rotary evaporator, dichloromethane is removed by distillation under reduced pressure to obtain the amphiphilic polylactic acid-hydroxyethyl starch-folate macromolecular compound-based nano drug-loading system containing the dimer compound drug.

In some embodiments, the dosage form of the nano drug-loading system is injection, powder injection, oral preparation, spray, capsule, or suppository.

In a preferred embodiment of the disclosure, by selecting hydroxyethyl starch as the hydrophilic segment, polylactic acid as the hydrophobic segment, and folic acid as the tumor-specific targeting molecule, combined with the emulsification solvent volatilization method, the drug-loaded nanoparticle loaded with drug CN, with a particle size of approximately 200 nm, and exhibiting uniform distribution and a stable structure is prepared. Compared with the nanoparticles without folic acid targeting, the nanoparticle significantly increases an enrichment speed and amount of the nanoparticles at the tumor site and promotes the uptake of the nanoparticles by tumor cells. In a variety of breast cancer 4T1 mouse models, better antitumor effects are shown, while toxic side effects are reduced, and the survival time of mice is prolonged. In the nano drug-loading system based on the polylactic acid-hydroxyethyl starch-folate macromolecular compound provided by the disclosure, the drug CN is obviously enhanced to induce the death of immunogenic cells (ICD) and inhibit the activity of indoleamine-2,3-oxygenase (IDO). Further, the maturation of lymph node dendritic cells (DC) is promoted, the number of effector memory T cells in the spleen increases, and the central memory T cells are maintained high. The tumor microenvironment is improved, the proportion of immunosuppressive T cells in the tumor is reduced, the content of tryptophan is increased, and the concentration of IL-6, IL-13, and TGF-β is reduced, so that the elimination of cancer stem cells is thereby achieved. The tumor is killed and the tumor immune microenvironment is also regulated, and the effect of tumor treatment is thereby enhanced, the combination of tumor chemotherapy and immunotherapy is achieved, and the antitumor effect is improved. Further, experiments have proved that its tumor suppression effect is better than that brought by the mature first-line drug Irinotecan and paclitaxel injection Taxol currently available on the market, and therefore, the nano drug-loading system provided by the disclosure exhibits good application prospects.

The disclosure further provides a novel method for removing stem cells. By inducing the death of immunogenic cells and simultaneously inhibiting indoleamine-2,3-oxygenase, the niches of cancer stem cells may be regulated in many ways, the dormancy of the cancer stem cells is relieved, the tumor-forming ability of the cancer stem cells is reduced, and the cancer stem cells and tumor cells are thus eliminated.

In an embodiment of the disclosure, a prodrug molecule CN and its nano dosage form PHF-CN are designed and prepared. Experiments have found that the nano dosage form may significantly induce immunogenic cell death, significantly inhibit the activity of indoleamine-2,3-oxygenase, significantly regulate various immune cells, cytokines, amino acids, etc. inside the tumor, improve the niche of cancer stem cells, relieve stem cell dormancy, eliminate cancer stem cells, and significantly inhibit tumor growth and prolong survival time.

Chemotherapeutic drugs are currently the main means of treating various tumors. However, traditional chemotherapeutic drugs cannot effectively kill cancer stem cells and may increase the stemness of tumor cells, induce cancer stem cells to enter dormancy, and reduce their response to drugs and immune cells. Some chemotherapeutic drugs may cause immunogenic cell death and activate antitumor immune responses. However, the activated immune cells are often hijacked by the inhibitory microenvironment after reaching the tumor site, making it difficult to exert antitumor effects.

Besides, the immunosuppressive microenvironment inside the tumor creates a cancer stem cell niche suitable for the growth and proliferation of cancer stem cells through immune cells, cytokines, and amino acids. Indoleamine 2,3-dioxygenase (IDO) plays an important role in maintaining the niche of cancer stem cells. IDO-mediated degradation of tryptophan to kynurenine is one of the important mechanisms for tumor cell immune escape. IDO catalyzes the metabolism of tryptophan, produces a large number of metabolites, enhances the stemness of tumor cells, induces cancer stem cells to enter dormancy, and reduces the sensitivity of cancer stem cells to tumor immunity, chemotherapy, and radiotherapy. IDO in tumors may also inhibit the activity of tumor-specific effector T cells, and at the same time, enhances the immunosuppressive activity and number of regulatory T cells (Treg).

The disclosure provides a method of eliminating a cancer stem cell, and the method may be applied to various tumor types and may be realized by various means. One or more of multiple methods such as chemotherapy, radiotherapy, hyperthermia, cryotherapy, magnetic therapy, sound waves, laser, and electrical stimulation may be used to cause immunogenic cell death. A variety of ways to inhibit indoleamine-2,3-oxygenase (IDO) are combined, including inhibition of IDO expression, inhibition of IDO activity, inhibition of IDO downstream pathways, and reduction of IDO metabolites, to enhance the immune response to tumors and improve the niche of cancer stem cells, thereby eliminating cancer stem cells and tumor cells.

Examples are provided as follows:

### Example 1

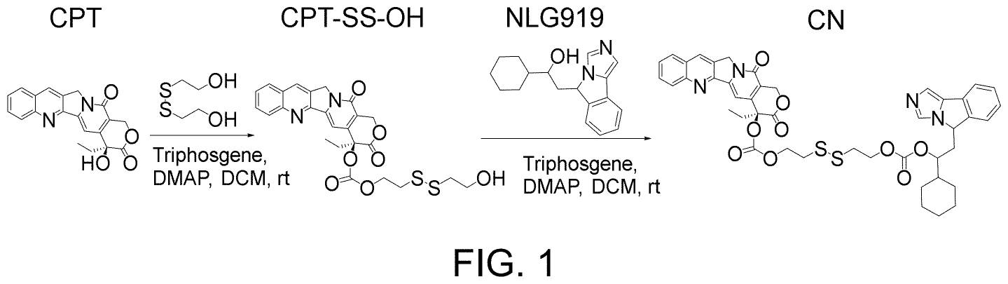

The compound CN represented by formula (2) was prepared according to the schematic flow chart as shown in FIG. 1, and proceeded as follows:

(1) Reaction of camptothecin with 2,2′-dithiodiethanol: camptothecin (1 mmol, 348.34 mg) and triphosgene (⅓ mmol, 98.91 mg) were dissolved in 20 ml of dichloromethane, 4-dimethylaminopyridine (1 mmol, 122.17 mg) was added, and after 30 minutes of reaction on ice and protected from light, 2,2′-dithiodiethanol (1 mmol, 154.25 mg) was added for reaction at room temperature and protected from light for 12 hours.

(2) The product obtained in (1) was extracted with 0.1 M aqueous hydrochloric acid, saturated brine, and ultrapure water to separate the organic phase, dried in a vacuum drying oven at 37° C. to remove the organic solvents, and dried.

(3) The product (1 eq) and triphosgene (⅓ eq) obtained in (2) were dissolved in dichloromethane, 4-dimethylaminopyridine (1 eq) was added, and after reaction for 30 minutes, NLG919 (1 eq) was added for reaction.

(4) The product obtained in (3) was extracted with 0.1M aqueous hydrochloric acid, saturated brine, and ultrapure water to separate the organic phase, dried to remove the organic solvent, and dried in a vacuum drying oven at 37° C. to obtain the crude product, which was purified by HPLC to obtain the compound CN.

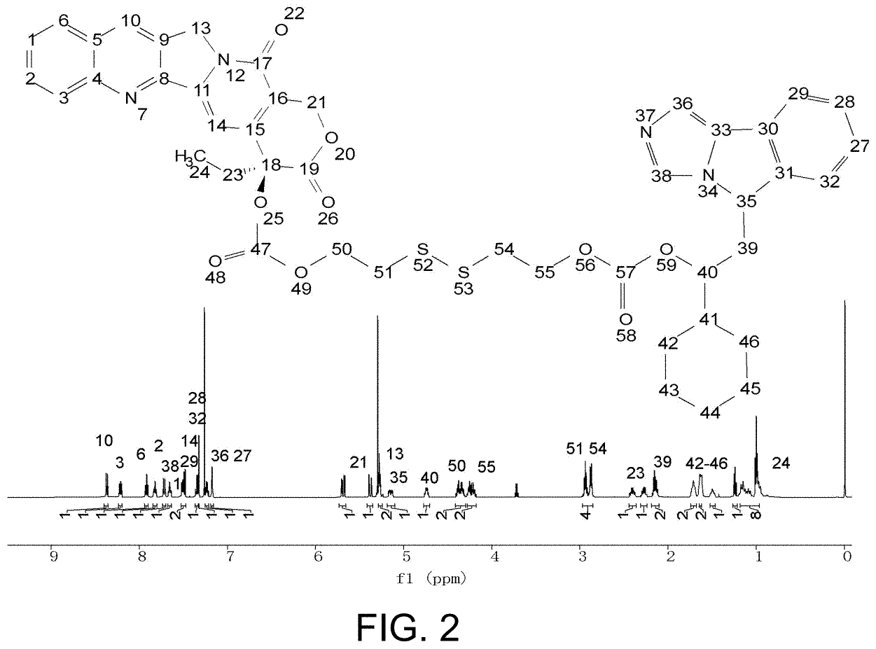

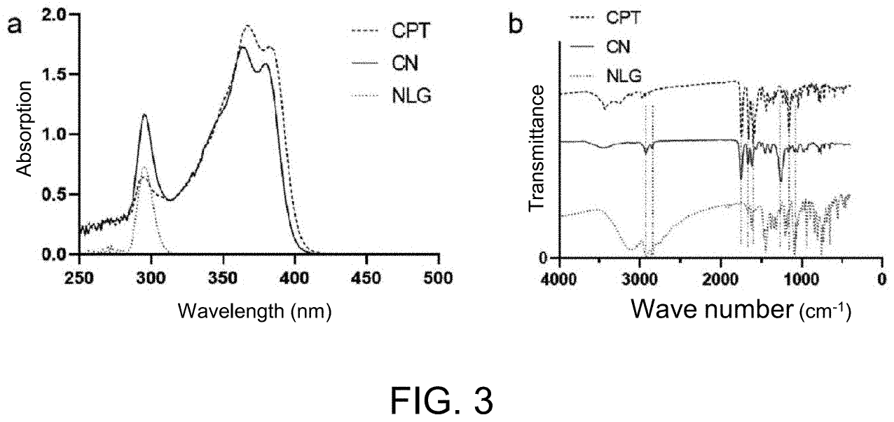

The compound obtained by separation and purification was tested by mass spectrometry, nuclear magnetic resonance, ultraviolet, infrared, and other data, so as to determine the structure of the compound. The ¹H NMR (600 MHz) data is shown in FIG. 2. The ultraviolet spectrum is shown in content a of FIG. 3, CN has both CPT absorption and

NLG919 absorption (where CPT stands for camptothecin, CN stands for the compound prepared in this example, and NLG stands for IDO inhibitor NLG919). The infrared spectrum is shown in content b of FIG. 3. CPT has the characteristic stretching vibration v(C-O) 1157 of tertiary alcohol, and NLG919 has the characteristic stretching vibration v(C-O) 1066 of primary alcohol. The two characteristic vibration peaks of CN disappear, and the characteristic stretch vibration v(C-O) 1255 of ester appears, indicating that CPT and NLG919 are connected to 2,2′-dithiodiethanol through an ester bond. HRESIMS [M+H]+ m/z 837.26047 (calcd for C₄₄H₄₄N₄O₉S₂, 837.26230) is a pale-yellow amorphous powder. It is illustrated that the compound CN represented by formula (2) was prepared in this example.

### Example 2

Study on the reduction responsiveness of compounds was performed as follows.

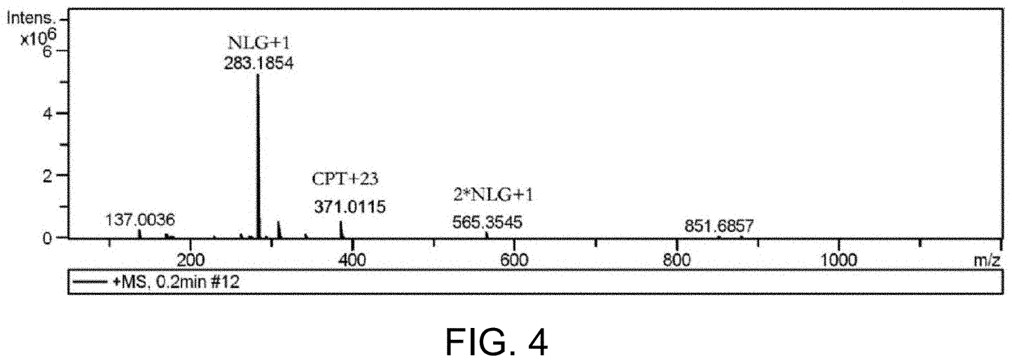

A small amount of the compound CN was added to 100 mM GSH aqueous solution and stirred for 12 hours. The stirred solution was analyzed by mass spectrometry, and the mass spectrometry result is shown in FIG. 4. The mass spectrometry result showed that the compound CN may be degraded to the original camptothecin and NLG919 under reducing conditions.

### Example 3

The inhibitory activity of the compound CN on IDO in vitro was investigated.

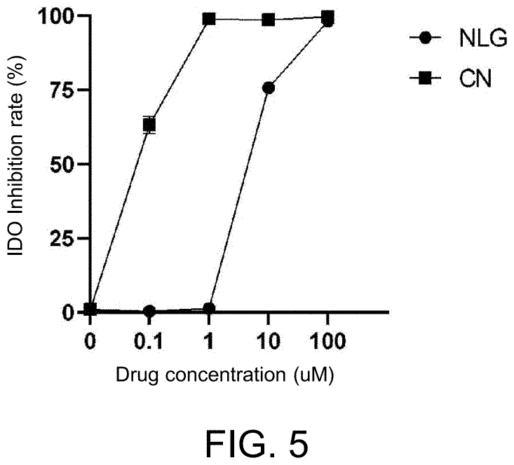

Hela cells were plated with a 96-well plate at 4,000 cells/well in DMEM complete medium containing 80 ng/ml of human IFN-γ, and incubated with a series of DMEM medium containing CN or NLG at a concentration of 0 μM, 0.1 μM, 1 μM, 10 μM, and 100 μM for 24 hours after 12 hours. 150 ul culture medium was aspirated, 75 ul trichloroacetic acid aqueous solution (30% w/v) was added, incubated at 50° C. for 30 min, and centrifuged at 3000 rpm for 10 minutes. 100 ul of the supernatant was collected, 100 ul of p-dimethylaminobenzaldehyde glacial acetic acid solution (2%, w/v) was added, placed at room temperature for 10 minutes, and the absorption was detected at 492 nm. The results are shown in FIG. 5.

It can be seen from FIG. 5 that the compound CN showed good IDO inhibitory activity in Hela cells. NLG919 needed 8 μM for the IDO inhibitory activity to reach 50%, while the dimer compound IDO inhibitory rate of this example only needed about 0.08 μM to reach 50%. Besides, when the concentration of the dimer compound in this example was about 1 μM, the IDO inhibition rate could be close to 100%.

### Example 4

The in vitro activity of the compound CN in inducing immunogenic cell death was evaluated.

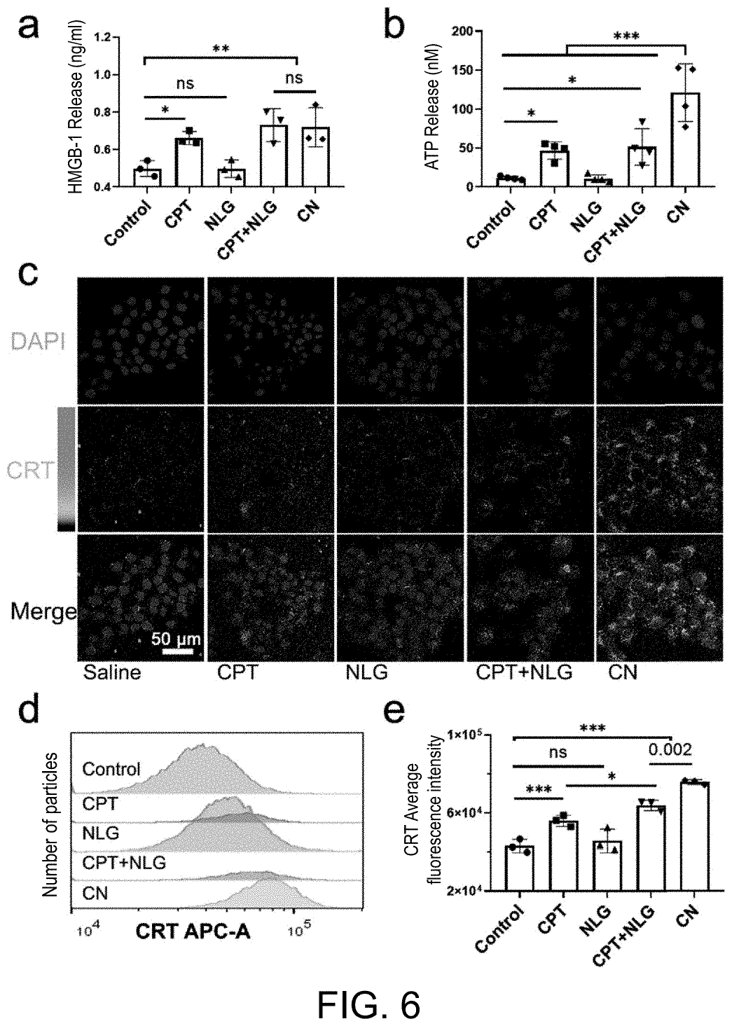

4T1 (breast cancer) cells were seeded into a 6-well plate at a density of 1×10⁵ cells per well and cultured overnight. The cells were treated with PBS, CPT (camptothecin), NLG (IDO inhibitor), CPT+NLG, CN (all drug concentrations were 10 μM) for 48 hours. The supernatant was collected and cultured to detect the release of ATP and HMGB-1. The cells were trypsinized, then incubated with CRT antibody on ice for 30 minutes in the dark, and analyzed by flow cytometry. In addition, CRT eversion was also studied by immunofluorescence imaging. 4T1 cells were seeded into a confocal culture dish at a density of 1×10⁵ cells per well and cultured overnight. The cells were treated with PBS, CPT (camptothecin), NLG (IDO inhibitor), CPT+NLG, CN (all drug concentrations were 10 μM) for 48 hours. The cells were washed twice with PBS and incubated with CRT antibody for 30 minutes. Washing was performed twice with PBS, and fixing was then performed with 4% paraformaldehyde. After washing was performed twice with PBS again, the nucleus was stained with DAPI for 30 minutes. After washing was performed twice, the cells were imaged with a confocal microscope. The results are shown in FIG. 6.

The results of content a in FIG. 6 show that the ability of CN to induce HMGB-1 is significantly stronger than that of the other groups. The results of content b in FIG. 6 show that CN-induced ATP release is about 2-3 times that of CPT and CPT+NLG groups. Content c of FIG. 6 is the result of confocal imaging, the result shows that CN can induce more CRT eversion.

Content d and content e of FIG. 6 are the results of flow cytometry analysis. The ability of CN group to induce CRT eversion was about 1.5 times that of CPT group. The above results prove that CN has a good ability to induce immunogenic cell death.

### Example 5

The anti-tumor activity of compound CN in vivo was studied.

In disclosure, the mouse 4T1 breast cancer orthotopic tumor model was used to investigate the anti-tumor effects of the compound CN, NLG919, camptothecin, and camptothecin combined with NLG919 injected intratumorally in mice. Specific steps are provided as follows.

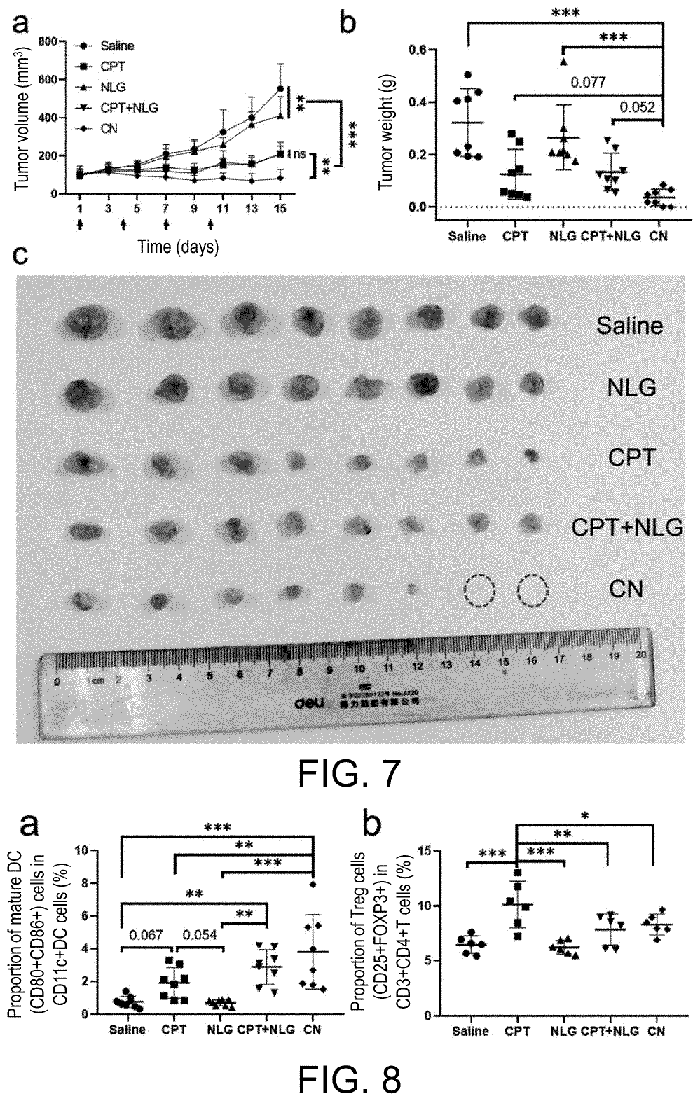

At the age of 6 weeks, 20 g female BALB/c mice were inoculated with 5×10⁵ cells of mouse breast cancer 4T1 cell suspension into the fourth pair of left breast pads on the abdomen of female BALB/c mice to establish a mouse model of 4T1 tumor in situ. When the tumor volume in situ was about 100 mm³, the mice were randomly divided into 5 groups, 8 in each group, and were given intratumoral injection of normal saline, intratumoral injection of free camptothecin, intratumoral injection of normal saline+gavage NLG, intratumoral injection of free camptothecin+gavage NLG, and intratumoral injection of free CN. During the treatment, the dose of camptothecin was 1 mg/kg, the dose of CN was 2.4 mg/kg (with the amount of 1 mg/kg of camptothecin and other substances), and the dose of NLG was 500 ug/mouse. The administration time on the first day was recorded as the first day, and then the above-mentioned doses were administered on the 4^{th}, 7^{th}, and 10^{th} days. From the 1^{st} day, the mouse body weight and tumor volume in situ were measured once a day, and the tumor volume-time curve was drawn. The mice were sacrificed on the 15th day, the tumor in situ was removed, weighed, and photographed. The results are shown in FIG. 7. In FIG. 7, saline represents normal saline, CPT represents camptothecin, NLG represents NLG919, and CPT+NLG represent camptothecin combined with NLG919.

Content a of FIG. 7 is the tumor volume of mice. It can be seen that the CN group could significantly inhibit the growth of the tumor, and the tumor volume was significantly smaller than that of the other groups. The tumor suppression rate of NLG was 25.4%, CPT was 61.9%, CPT+NLG was 62.1%, and the tumor suppression rate of CN was the highest at 85%. Content b is the mass of the exfoliated tumor, and it can be seen that the weight of the tumor in the CN group was significantly lower than that of the other groups. Content c is a picture of the tumor stripped after the experiment. It can be seen that the tumor in the CN group was significantly smaller than the other groups, and one tumor was completely eliminated. The above results indicate that intratumoral injection of free CN has achieved better antitumor effects than other administration groups.

### Example 6

The effect of the compound CN on antitumor immune response is studied, the specific steps were proceeded as follows.

The tumor and adjacent lymph nodes of the mice in Example 4 were stripped. The tumor was stripped, cut with ophthalmic scissors, and incubated in 1640 medium with collagenase 1 and DNase dissolved for 60 minutes at 37° C., and then squeezed through a 200-mesh screen to prepare a single cell suspension for immunoregulatory T cells (CD3, CD4, CD25, FoxP3) at the tumor site. The adjacent lymph nodes were squeezed through a 200-mesh screen to prepare a single cell suspension, and the maturation of lymph node dendritic cells (DC cells, CD11c, CD80, CD86) was studied with antibodies. The results are shown in FIG. 8.

Content a of FIG. 8 is the effect of drugs on the maturation of dendritic cells in the lymph nodes adjacent to tumors.

After combined with IDO inhibitor, the DC maturation of CPT+NLG group and CN group increased significantly, and the proportion of mature DC in CN group was about twice that of CPT group. Content b of FIG. 8 shows the effect of drugs on regulatory T cells (Treg) in tumors. The results show that the chemotherapy drug CPT can cause a significant increase in Treg in tumors, but the CPT+NLG and CN groups combined with IDO inhibitors can reverse the increase in Treg caused by CPT. The compound CN inhibits IDO activity by inducing immunogenic cells, which not only increases DC maturation, but also weakens Treg cell-mediated immunosuppression, and enhances anti-tumor immune response from both positive and negative aspects.

### Example 7

The effect of the compound CN on cancer stem cells is studied, the specific steps were proceeded as follows.

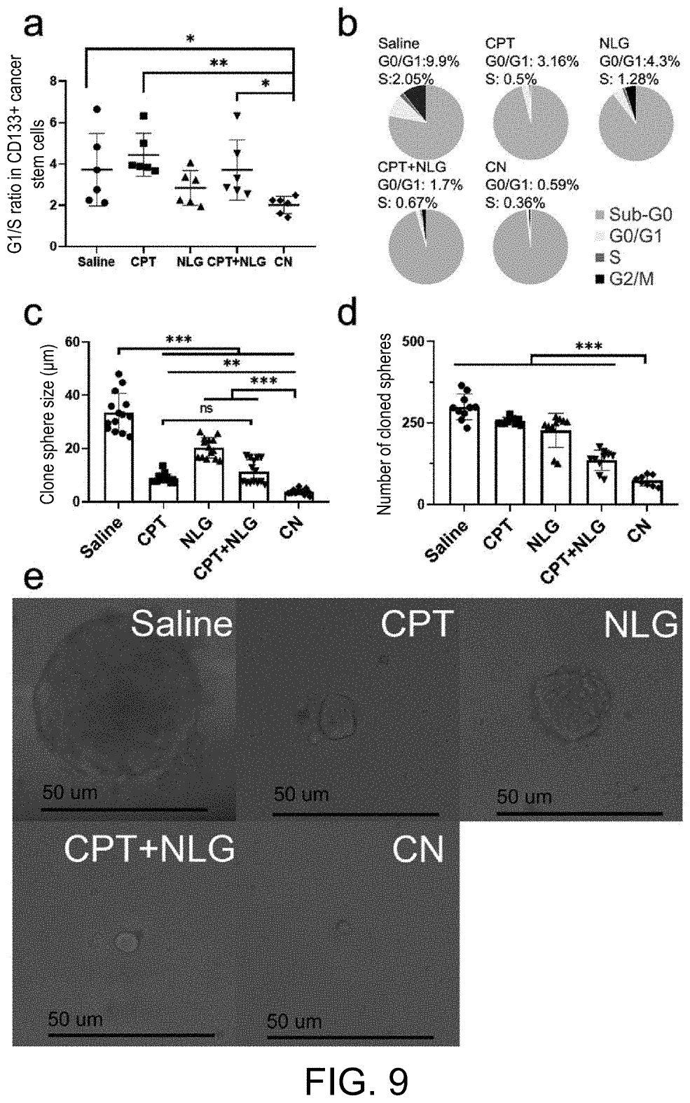

In the single cell suspension prepared in Example 6, the cell cycle (CD133, EdU, hoechst33342) of cancer stem cells was stained with antibodies, and the dormancy of the cancer stem cells was analyzed by flow cytometry. Part of the single cell suspension was placed in RPMI1640 medium containing 10% fetal bovine serum, and the cell concentration was adjusted to 1.6×10⁴ cells/mL. Fibrinogen was diluted to 20 mg/mL with T7 buffer (pH 7.4, 50 mM Tris, 150 mM NaCl), and then mixed with the above cell suspension in equal volume. 1 μL of thrombin (0.1 U/μL) was added to the 96-well plate in advance, and 50 μL of the mixed cell suspension was added. After incubating for 30 minutes at 37° C., 200 μL of RPMI1640 containing 10% fetal bovine serum and 1% double antibody were added to the culture medium to continue the culture. On the seventh day, the cell pellets in the 96-well plate were photographed and counted. The particle size of tumor cell spheroids was measured, and the number of tumor cell spheroids was counted to characterize the stemness and tumor-forming ability of tumor cells. The results are shown in FIG. 9.

Content a and content b of FIG. 9 show that the chemotherapeutic drug CPT can block the G1-S cell cycle of cancer stem cells and make cancer stem cells dormant. The CN and CPT+NLG groups combined with IDO inhibitors can significantly reduce the G1/S ratio, relieve cancer stem cell dormancy, enable cancer stem cells to enter the S phase, and enhance the sensitivity of cancer stem cells to chemotherapeutic drugs and immune cells. The result of content b in FIG. 9 also shows that the cancer stem cells in the CN group are in the Sub-G0 stage most, indicating that the CN group can effectively kill the cancer stem cells. As shown in content c, content d, and content e of FIG. 9, the number and particle size of 3D spheres of tumor cells in the CN group were significantly lower than those in the other groups, indicating that the stemness of the tumor cells remaining in the tumor site after CN treatment was significantly reduced, the tumor-forming ability was significantly reduced, and the stem cells were effectively eliminated. In summary, CN effectively relieved cancer stem cell dormancy, killed cancer stem cells, and reduced the stemness and tumor-forming ability of residual tumor cells at the tumor site.

### Example 8

Preparation of Compound PHF

The preparation of the compound PHF represented by formula (3) was carried out according to the following steps.

(1) Folic acid (20 mmol, 8.828 mg) and dicyclohexylcarbodiimide (0 mmol, 8.253 mg) were dissolved in 10 mL of dimethyl sulfoxide, 4-dimethylaminopyridine (40 mmol, 4.887 mg) was added, and the reaction was carried out at 40° C. for 30 minutes.

(2) Hydroxyethyl starch (1 mmol, 130 mg) was dissolved in 5 mL dimethyl sulfoxide, added to the reaction system in (1), and the reaction was continued at 40° C. for 48 hours.

(3) The reaction solution in (2) was added to 100 ml of ethanol to precipitate, centrifuged at 8,000 rpm for 10 minutes, the precipitate was dissolved with dimethyl sulfoxide, placed in a dialysis bag (Mw 8000 Da), dialyzed with ultrapure water for two days, and freeze-dried to obtain pure FA-HES.

(4) Polylactic acid (4 mmol, 32 mg) and dicyclohexylcarbodiimide (8 mmol, 1.651 mg) were dissolved in 10 mL dimethyl sulfoxide, 4-dimethylaminopyridine (8 mmol, 0.977 mg) was added, and the reaction was carried out at 40° C. for 30 minutes.

(5) FA-HES (1 mmol, 130 mg) was dissolved in 5 mL dimethyl sulfoxide, added to the reaction system in (4), and the reaction was continued at 40° C. for 48 hours.

(6) The reaction solution in (5) was added to 100 ml ethanol to precipitate, centrifuged at 8,000 rpm for 10 minutes to precipitate, dissolved with dimethyl sulfoxide, placed in a dialysis bag (Mw 8000 Da), dialyzed with ultrapure water for two days, then freeze-dried to obtain pure the compound PHF as shown in formula (3). The molecular weight of polylactic acid was 8 KDa, the molecular weight of hydroxyethyl starch was 130 KDa, the grafting rate of polylactic acid on hydroxyethyl starch was 0.86, and the grafting rate of folic acid on hydroxyethyl starch was 20.

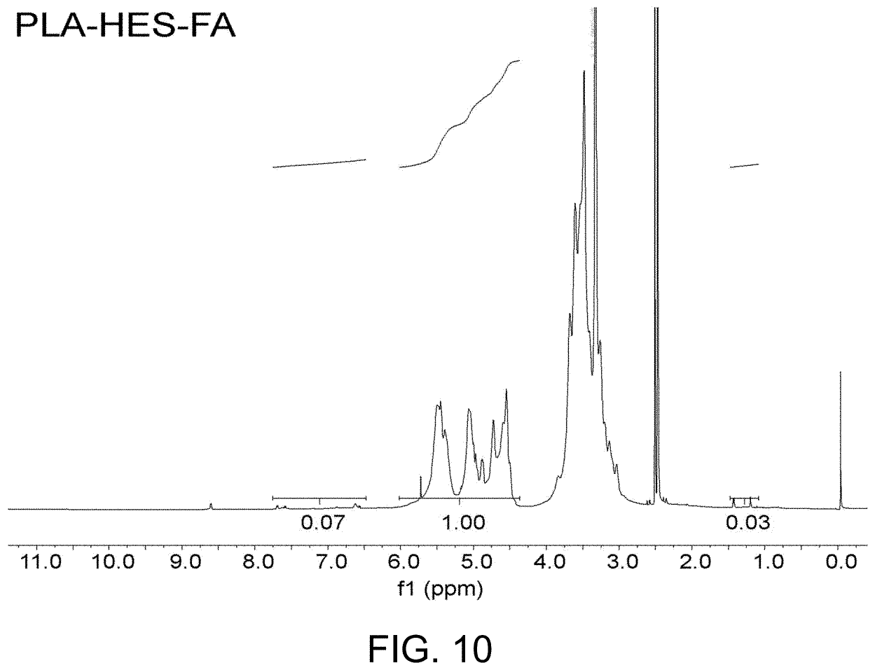

The compound obtained by separation and purification was subjected to nuclear magnetic resonance test to determine the structure of the compound. The ¹H NMR data is shown in FIG. 10. There is a proton peak of the hydroxyl group on the glucose ring of hydroxyethyl starch on the proton nuclear magnetic resonance spectrum of PHF, that is, a multiple peak with a chemical shift between 4.5 ppm and 6 ppm. The methyl proton peak of polylactic acid with a chemical shift between 1 ppm to 2 ppm can also be found, and the proton peak on the benzene ring of folic acid with a chemical shift between 6 ppm and 8 ppm can also be found. Through the NMR spectrum, it was confirmed that PLA-HES-FA has been successfully prepared.

A CN@PHF nano-preparation containing CN was prepared and characterized.

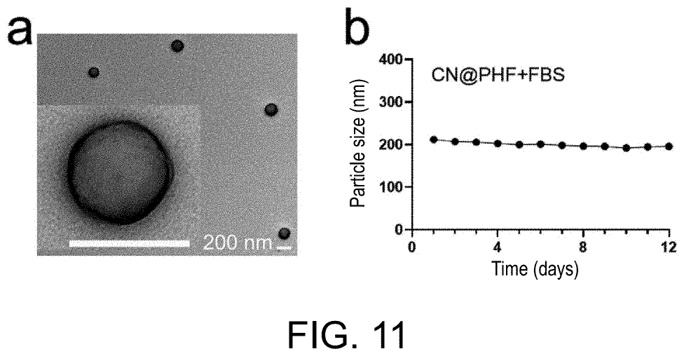

The polylactic acid-hydroxyethyl starch-folate macromolecular compound (PLA-HES-FA) was selected as the nano carrier. PHF (100 mg) was dissolved in 5 mL ultrapure water and placed on ice. The drug CN (20 mg) was dissolved in 0.25 mL of dichloromethane, slowly added dropwise to the PHF aqueous solution, and ultrasonic emulsified while adding dropwise. The obtained emulsion was removed with a rotary evaporator to remove dichloromethane, placed in a dialysis bag (Mw 3500 Da), dialyzed with ultrapure water for one day, and then freeze-dried to obtain drug-loaded nanoparticles CN@PHF. 1 mg/mL lyophilized powder aqueous solution was prepared, ultrasonicated for 10 minutes, and then set aside. In the volume of 1 mL, a laser particle size analyzer (Nano-ZS90, Malvern) was used to detect the hydrated particle size, particle size distribution, and potential of CN@PHF. The measurement temperature was 25° C., and the laser light source: He-Ne laser, the wavelength was 633 nm. The results are shown in Table 1. 20 uL of 1 mg/ml nano-particle CN@PHF dispersion was dropped on a copper net, dyed with 0.1% phosphotungstic acid, and dried naturally at room temperature, and its morphology was observed with a transmission electron microscope (TEM, H-700FA, HITACHI). The acceleration voltage was 20 KV to 125 KV. CN@PHF was added to a PBS solution containing 10% fetal bovine serum for 10 consecutive days. DLS was used to detect the particle size of the nanoparticles every day to evaluate the stability of the nanoparticles. The results are shown in FIG. 11. The drug loading of CN in CN@PHF was detected by ultraviolet spectrophotometry. CN@PHF was weighed to obtain the mass W₁ (mg). The mass of CN in CN@PHF was W₂ (mg) measured by UV spectrophotometry, and the drug loading was based on the formula DLC (%)=W₂/W₁×100%. Encapsulation rate EE (%)=W₂/20×100%.

The TEM result of content a in FIG. 11 shows that the particle size of CN@PHF is about 170 nm, which is consistent with the result of content b in FIG. 11 that the hydrated particle size of CN@PHF is 200 nm measured by DLS. Moreover, in the simulated blood environment, the drug-loaded nanoparticle CN@PHF has no significant change in particle size within 10 days, indicating that CN@PHF has good stability and is beneficial to its long circulation in the blood.

TABLE 1
Characterization of physical and chemical
properties of nanoparticles

| | | Particle size | | | |
|---|---|---|---|---|---|
| | Particle size | distribution | ζ potential | DLC | EE |
| Nanoparticles | (nm) | PDI | (mV) | (%) | (%) |
| CN@PHF | 209.5 | 0.13 | 0.07 | 14.6 | 87.6 |

### Example 9

The anti-tumor activity of CN@PHF in the mouse 4T1 breast cancer orthotopic tumor model was investigated and the specific steps were proceeded as follows:

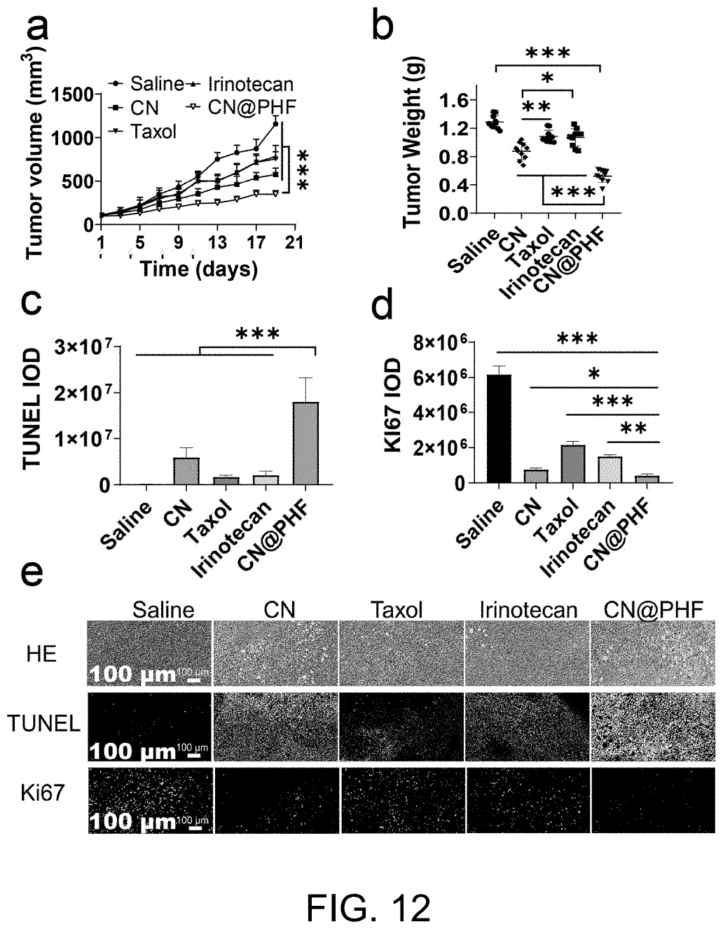

At the age of 6 weeks, 20 g female BALB/c mice were inoculated with 5×10⁵ cells of mouse breast cancer 4T1 cell suspension into the fourth pair of left breast pads on the abdomen of female BALB/c mice to establish a mouse model of 4T1 tumor in situ. When the tumor volume in situ was about 100 mm³, the mice were randomly divided into 5 groups, 10 mice in each group, and were injected with normal saline, CN, Taxol, Irinotecan, CN@PHF through the tail vein. The dose of CN was 2.4 mg/kg, and the concentrations of Taxol and Irinotecan were the same as the molar concentration of CN. The administration time on the first day was recorded as the first day, and then the above-mentioned doses were administered on the 4^{th}, 7^{th}, and 10^{th} days. From the 1^{st} day, the mouse body weight and tumor volume were measured every other day, and the tumor volume-time curve was drawn. The mice were sacrificed on the 19th day, the tumor in situ was removed, weighed, and photographed. Tumors were stained with HE and TUNEL to study tumor cell apoptosis and stained with Ki67 to study tumor cell proliferation. The results are shown in FIG. 12.

Content a of FIG. 12 is the tumor volume of mice, and content b of FIG. 12 is the tumor weight. It can be seen that both CN@PHF and CN groups can significantly inhibit tumor growth, and the tumor volume is significantly smaller than the normal saline group and the positive drugs Taxol and Irinotecan groups. The tumor volume and weight of mice in the CN@PHF group are significantly smaller than those of the other groups, showing the strongest anti-tumor activity. Content c, content d, and content e of FIG. 12 are colored HE, TUNEL, and Ki67 respectively. It can be seen that the necrotic area of the tumor site after CN@PHF treatment was larger than that of the other groups, the tumor cell apoptosis was significantly higher than that of the other groups, the proliferation of tumor cells was significantly lower than that of the other groups, and the slice data further confirmed that CN@PHF showed a better anti-tumor effect. In summary, both CN@PHF and free CN showed good antitumor activity in the mouse 4T1 breast cancer orthotopic tumor model, due to the traditional first-line chemotherapeutics Taxol and Irinotecan.

### Example 10

The mouse 4T1 breast cancer orthotopic tumor model was used to investigate the effect of CN@PHF on tumor immune response and the elimination of cancer stem cells, and the specific steps were proceeded as follows:

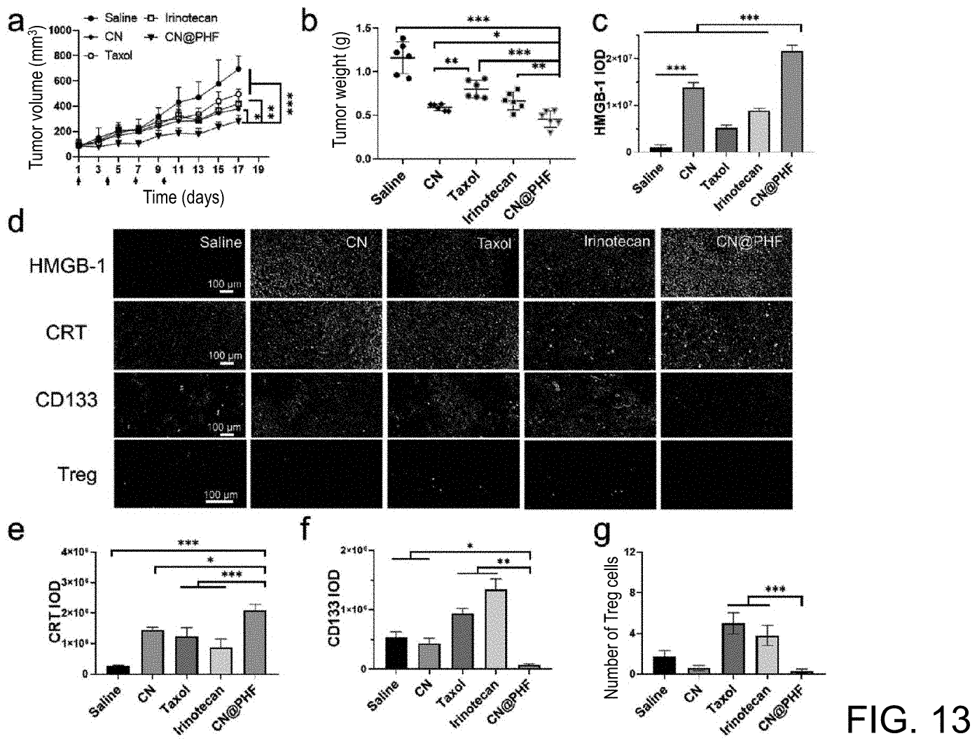

At the age of 6 weeks, 20 g female BALB/c mice were inoculated with 5×10⁵ cells of mouse breast cancer 4T1 cell suspension into the fourth pair of left breast pads on the abdomen of female BALB/c mice to establish a mouse model of 4T1 tumor in situ. When the tumor volume in situ was about 100 mm³, the mice were randomly divided into 5 groups, 6 mice in each group, and were injected with normal saline, CN, Taxol, Irinotecan, CN@PHF through the tail vein. The dose of CN was 2.4 mg/kg, and the concentrations of Taxol and Irinotecan were the same as the molar concentration of CN. The administration time on the first day was recorded as the first day, and then the above-mentioned doses were administered on the 4^{th}, 7^{th}, and 10^{th} days. From the 1^{st} day, the mouse body weight and tumor volume were measured every other day, and the tumor volume-time curve was drawn. The mice were sacrificed on the 17^{th} day, and the tumor in situ, the lymph nodes adjacent to the tumor, and the spleen were removed. Tumors were stained with immunofluorescence, HMGB-1, and CRT staining to study the ability of drugs to cause immunogenic cell death at the tumor site. CD4 and FoxP3 were co-stained to study the amount of Treg that inhibits antitumor immunity at the tumor site, and CD133 staining was used to evaluate the amount of cancer stem cells at the tumor site. The results are shown in FIG. 13.

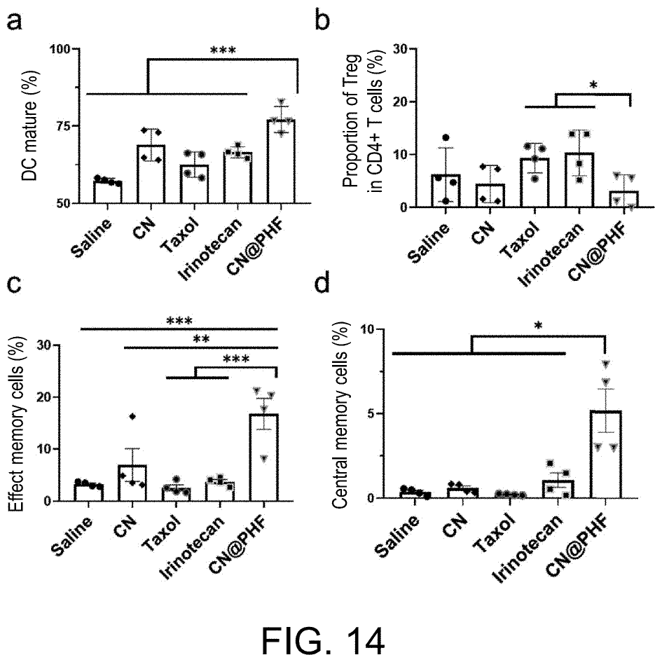

The stripped spleen and tumor were cut with ophthalmic scissors and incubated in 1640 medium with collagenase 1 and DNase dissolved at 37° C. for 60 minutes, and then squeezed through a 200-mesh screen to prepare a single cell suspension. The lymph nodes adjacent to the tumor were squeezed through a 200-mesh screen to prepare a single cell suspension. Flow cytometric analysis was performed on the maturation (CD11c, CD80, CD86, MHC-II) of lymph node dendritic cells (DC), immune regulatory T cells (CD4, CD25, FoxP3) at the tumor site, and immune memory T cells (CD3, CD8, CD44, CD62L) in the spleen. The effect of drugs on tumor immunity was investigated. The results are shown in FIG. 14.

Content a and content b of FIG. 13 show that CN@PHF showed the best antitumor activity in the 4T1 orthotopic tumor model of triple-negative breast cancer mice. In FIG. 13, content d is an immunofluorescence section, content c, content e, content f, and content g are the quantitative data statistics of HMGB-1, CRT, CD133, and Treg, respectively. The results show that CN@PHF has the strongest ability to induce the death of immune cells, which is manifested in that CN@PHF can induce higher HMGB-1 release and CRT eversion at the tumor site. As shown in content d and content g of FIG. 13, CN@PHF significantly inhibits the amount of Treg immune regulatory T cells at the tumor site. As shown in content d and content f of FIG. 13, CN@PHF has realized the efficient elimination of cancer stem cells at the tumor site.

Content a of FIG. 14 shows that CN@PHF significantly promotes the maturation of DC cells in the lymph nodes adjacent to tumors. Content b of FIG. 14 shows that CN@PHF significantly inhibits the proportion of Treg cells in the tumor site in CD4+ T cells. Content c and content d of FIG. 14 show that CN@PHF effectively triggers an anti-tumor immune memory response, significantly increases the content of effector memory T cells, while maintaining a high content of central memory T cells.

The above results show that CN@PHF can activate the maturation and antigen presentation of dendritic cells (DC) in the adjacent lymph nodes by inducing the death of immune cells, promote the anti-tumor immune response, and trigger the anti-tumor immune memory. Moreover, CN@PHF suppresses the immune regulatory T cells (Treg) at the tumor site, thereby lifting the immune suppression of the tumor microenvironment and further enhancing the anti-tumor immune response. In the end, effective killing of cancer stem cells is achieved.

### Example 11

The mouse 4T1 breast cancer orthotopic tumor model was used to investigate the influence of CN@PHF on the niche of cancer stem cells, and the specific steps were proceeded as follows:

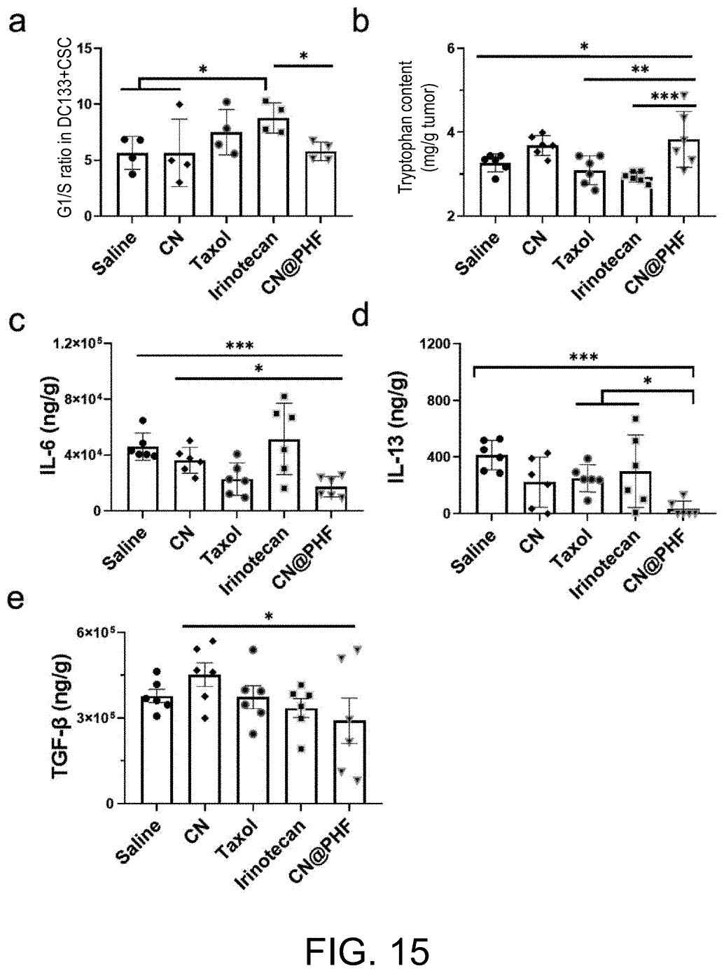

At the age of 6 weeks, 20 g female BALB/c mice were inoculated with 5×105 cells of mouse breast cancer 4T1 cell suspension into the fourth pair of left breast pads on the abdomen of female BALB/c mice to establish a mouse model of 4T1 tumor in situ. When the tumor volume in situ was about 100 mm³, the mice were randomly divided into 5 groups, 10 mice in each group, and were injected with normal saline, CN, Taxol, Irinotecan, CN@PHF through the tail vein. The dose of CN was 2.4 mg/kg, and the concentrations of Taxol and Irinotecan were the same as the molar concentration of CN. The administration time on the first day was recorded as the first day, and then the above-mentioned doses were administered on the 4^{th}, 7^{th}, and 10^{th} days. On the 13th day, 50 μL of Edu (8 mg/ml) was injected into the tumor. Three hours later, the mice were sacrificed, the tumor in situ was stripped and divided into two parts. One portion was cut with ophthalmic scissors and incubated in 1640 medium with collagenase 1 and DNase dissolved at 37° C. for 60 minutes, and then squeezed through a 200-mesh screen to prepare a single cell suspension. Flow cytometric analysis of the cell cycle (CD133, EdU, hoechst33342) of cancer stem cells was carried out to investigate the effect of drugs on the dormancy of cancer stem cells. As for the other portion, 500 μL PBS and 400 μL methanol were added, homogenized, centrifuged, and the supernatant was collected, and the tryptophan content in the supernatant was detected by HPLC. The results are shown in FIG. 15.

As shown in content a of FIG. 15, the chemotherapeutic drugs used in the first-line clinic, such as Taxol and Irinotecan, may increase the dormancy of cancer stem cells and lead to cycle inhibition. CN@PHF can relieve this cycle inhibition, thereby effectively eliminating cancer stem cells. Content b of FIG. 15 shows that CN@PHF can effectively inhibit the activity of indoleamine 2,3-oxygenase (IDO), thereby maintaining a higher tryptophan content in the tumor site. In addition, as shown in content c, content d, and content e of FIG. 15, CN@PHF significantly reduces the content of immunosuppressive cytokines IL-6, IL-13, and TGF-β at the tumor site. By maintaining the inhibition of IDO, CN@PHF maintains a higher concentration of tryptophan in the tumor site and reduces the content of immunosuppressive cytokines, thereby improving the entire cancer stem cell niche, making it no longer conducive to the growth and proliferation of cancer stem cells and tumor cells.

### Example 12

The mouse 4T1 breast cancer in situ tumor model was used to investigate the effect of CN@PHF on the survival time of mice, and the specific steps were proceeded as follows:

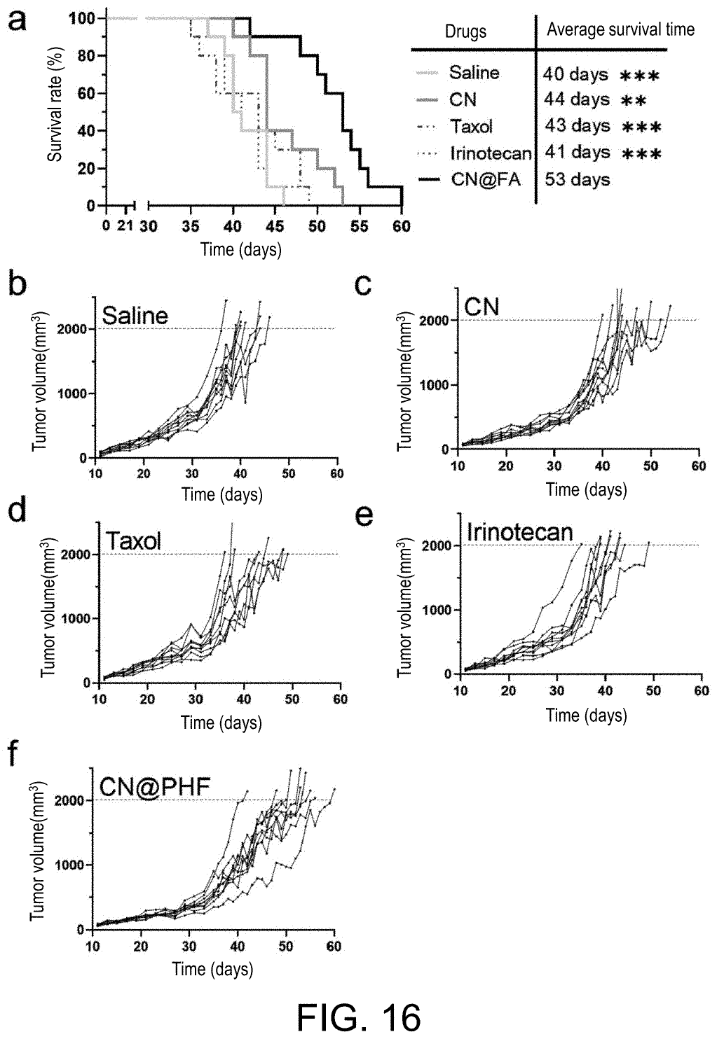

At the age of 6 weeks, 20 g female BALB/c mice were inoculated with 5×10⁵ cells of mouse breast cancer 4T1 cell suspension into the fourth pair of left breast pads on the abdomen of female BALB/c mice to establish a mouse model of 4T1 tumor in situ. When the tumor volume in situ was about 100 mm³, the mice were randomly divided into 5 groups, 10 mice in each group, and were injected with normal saline, CN, Taxol, Irinotecan, CN@PHF through the tail vein. The dose of CN was 2.4 mg/kg, and the concentrations of Taxol and Irinotecan were the same as the molar concentration of CN. The administration time on the first day was recorded as the first day, and then the above-mentioned doses were administered on the 4^{th}, 7^{th}, and 10^{th} days. From the 1^{st} day, the mouse body weight and tumor volume were measured every other day, and the tumor volume-time curve was drawn. When the tumor volume of the mouse exceeded 2,000 mm³, it was considered dead, and it was euthanized. The survival period of mice was recorded. The experimental results are shown in FIG. 16.

In FIG. 16, content a, content b, content c, content d, content e, and content f show that CN@PHF can significantly increase the average survival time of mice with triple-negative breast cancer. Compared with the normal saline group, the positive drug Taxol, Irinotecan group, and the free drug CN group, CN@PHF prolonged the average survival time of mice by about 10 days.

A person having ordinary skill in the art should be able to easily understand that the above description is only preferred embodiments of the disclosure and is not intended to limit the disclosure. Any modifications, equivalent replacements, and modifications made without departing from the spirit and principles of the disclosure should fall within the protection scope of the disclosure.

What is claimed is:

1. A camptothecin-based dimer compound, comprising a structural formula as shown in formula (1): 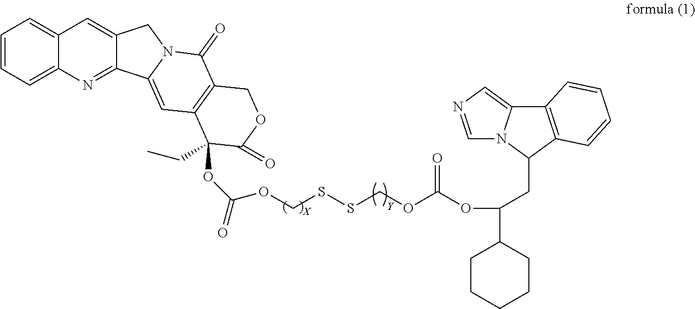
where x is an integer of 1 to 4, and y is an integer of 1 to 4.

2. An anticancer drug, comprising the dimer compound according to claim 1 and a pharmaceutically acceptable additive.

3. The anticancer drug according to claim 2, wherein a dosage form of the anticancer drug is injection, powder injection, oral preparation, spray, capsule, or suppository.

4. The anticancer drug according to claim 2, further comprising a nano drug-loading system and the pharmaceutically acceptable additive,
wherein the nano drug-loading system comprises a drug carrier and an antitumor drug, the antitumor drug is the dimer compound, the drug carrier comprises a polylactic acid-hydroxyethyl starch-folate macromolecular compound, and the macromolecular compound is obtained by coupling folic acid and polylactic acid with hydroxyethyl starch through an ester bond and has a structural formula shown in formula (3): 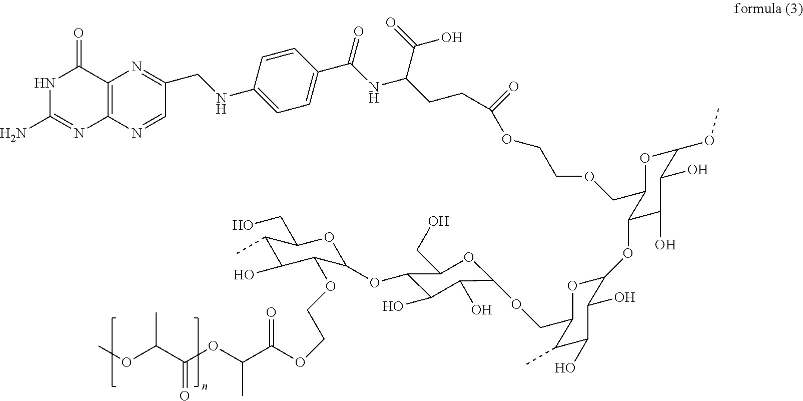
wherein a molecular mass of the polylactic acid is 6 KDa to 10 KDa, a molecular mass of the hydroxyethyl starch is 100 KDa to 150 KDa, a grafting ratio of the polylactic acid on the hydroxyethyl starch is 0.5 to 1, and a grafting ratio of the folic acid on the hydroxyethyl starch is 10 to 30.

5. The anticancer drug according to claim 4, wherein in the structural formula of the dimer compound, x=2 and y=2.

6. The anticancer drug according to claim 4, wherein a size of a drug-loaded nanoparticle of the nano drug-loading system is 100 nm to 200 nm.

7. The anticancer drug according to claim 5, wherein a size of a drug-loaded nanoparticle of the nano drug-loading system is 100 nm to 200 nm.

8. The anticancer drug according to claim 4, wherein a preparation method of the nano drug-loading system comprises the following steps:
(1) dissolving the amphiphilic polylactic acid-hydroxyethyl starch-folate macromolecular compound in water, performing ultrasonic emulsification in an ice bath, and simultaneously adding an organic solution in which the dimer compound shown in formula (1) is dissolved to obtain an ultrasonic emulsion;
(2) removing an organic solvent of the emulsion obtained in step (1) through reduced pressure distillation to obtain the amphiphilic polylactic acid-hydroxyethyl starch-folate macromolecular compound-based nano drug-loading system containing the dimer compound drug.

9. A method for removing a cancer stem cell and a tumor cell, comprising relieving dormancy of cancer stem cells through improving a cancer stem cell niche to eliminate the cancer stem cells and improve antitumor efficacy;
inducing immunogenic cell death and inhibiting indoleamine-2,3-oxygenase to improve the tumor stem cell niche;
performing one or more of chemotherapy, radiotherapy, hyperthermia, cryotherapy, magnetic therapy, sound waves, laser, and electrical stimulation to induce immunogenic cell death; and
performing one or more of the following: inhibiting expression of indoleamine-2,3-oxygenase, inhibiting activity of indoleamine-2,3-oxygenase, inhibiting a downstream pathway of indoleamine-2,3-oxygenase, and reducing a metabolite of indoleamine-2,3-oxygenase by using an anticancer drug to inhibit an indoleamine-2,3-oxygenase pathway and to improve the tumor stem cell niche and ultimately eliminate the tumor stem cell and the tumor cell;
wherein the anticancer drug comprises the camptothecin-based dimer compound according to claim 1.

10. The method according to claim 9, wherein the cancer is breast cancer, liver cancer, colon cancer, ovarian cancer, or melanoma.
